(19) United States
(12) Patent Application Publication
(10) Pub. No.: US 2005/0165272 A1
(43) Pub. Date: Jul. 28, 2005

(54) Endoscope system

(76) Inventors: Yuta Okada, Hachioji-shi (JP); Yukio Nakajima, Oyster Bay, NY (US)

Correspondence Address: OSTROLENK FABER GERB & SOFFEN, 1180 AVENUE OF THE AMERICAS, NEW YORK, NY, 100368403, US

(21) Appl. No.: 11/001,353

(22) Filed: Dec. 1, 2004

Related U.S. Application Data
(60) Provisional application No. 60/526,368, filed on Dec. 1, 2003.

(51) Int. Cl. A61B 1/00

(52) U.S. Cl. 600/114; 600/104; 600/113

(57) ABSTRACT

A endoscope system having an insertion section to be inserted into a body cavity, an introducer for performing treatment in the body cavity, and a capsule endoscope. The capsule endoscope has an observation optical system and is used in combination with the introducer.

### CROSS-REFERENCE TO RELATED APPLICATIONS

This application claims the benefit of U.S. Provisional Application No. 60/526,368, filed Dec. 1, 2003.

### BACKGROUND OF THE INVENTION

1. Field of the Invention

The present invention relates to an endoscope system for use in a surgical procedure by being inserted into a body cavity of a human subject.

2. Description of the Related Art

As an endoscope device for use in the observation of a living body tissue by being inserted into a body cavity of a human being, such a type is disclosed in, for example, in Jpn. Pat. Appln. KOKAI Publication Nos. 7-275195 and 7-275196. Here, a storage section of an image pickup section is provided at a distal end of an insertion section of the endoscope. In the storage section, the image pickup section is stored in such a way as to allow the image pickup section to be removed from the rest of the endoscope. An illumination optical system, treating tool insertion channel, water/air supply channel, etc., are provided in the insertion section of the endoscope.

PCT National Publication No. 2001-526072 discloses an endoscope system in which a clamping mechanism is provided at the distal end of the insertion section of the endoscope to allow a capsule which is used for measuring various kinds of measurements, etc., as well as for the administration of a drug, to be releasably clamped at the distal end of the insertion section of the endoscope.

### BRIEF SUMMARY OF THE INVENTION

An endoscope system according to the present invention comprises: a treating device which is configured to perform a treatment in a body cavity and which has an insertion section to be inserted into the body cavity through an opening to the body cavity, the insertion section having a distal end portion and a proximal end portion; and an observation device which has at least an outer case and an observation optical system and which is used in combination with the treating device.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a diagrammatic view generally showing an endoscope system as a whole according to a first embodiment of the present invention;

FIG. 2 is a longitudinal cross-section view showing an inner arrangement of the capsule endoscope of the endoscope system according to a first embodiment;

FIG. 3A is a view showing a state in which the capsule endoscope is engaged with the distal end of the endoscope when the capsule endoscope of the endoscope system of the first embodiment is detained in a body cavity of a patient;

FIG. 3B is a view showing a state in which the capsule endoscope is dropped from the distal end of the endoscope of the endoscope system according to the present invention;

FIG. 4 is a perspective view showing a state in which the capsule endoscope of the endoscope system of the first embodiment is inserted into the body cavity in a way to align with the distal end of the endoscope;

FIG. 5 is a perspective view showing a state in which the capsule endoscope of the endoscope system of the first embodiment is clipped by extending the clipping forceps from the distal end of the endoscope and fixed it in place;

FIG. 6 is a perspective view showing an outer appearance of an introducer, as a whole, of the endoscope system according to the first embodiment;

FIG. 7 is a longitudinal cross-section view showing a distal end portion of the introducer of the endoscope system of the first embodiment;

FIG. 8 is a perspective view showing a state in which the capsule endoscope of the endoscope system of the first embodiment is used in combination with the introducer;

FIG. 9 is a perspective view showing a state in which a narrower-size endoscope is inserted into an overtube of the endoscope system of the first embodiment;

FIG. 10A is a perspective view showing a state in which an introducer of an endoscope system of a second embodiment of the present invention is introduced into a body cavity of a patient with a capsule endoscope coupled to the introducer;

FIG. 10B is a perspective view showing a state in which a capsule endoscope is dropped from the introducer;

FIG. 11 is a perspective view for explaining a recovery of the capsule endoscope of the endoscope system of the second embodiment;

FIG. 12 is a perspective view showing a major portion of a capsule endoscope of an endoscope system of a third embodiment of the present invention which is moved to a position where a treating tool insertion bore of an introducer is opened;

FIG. 13 is a perspective view showing a major portion of the capsule endoscope of the endoscope system of the third embodiment which is moved to a closed position of the treating tool insertion hole of the introducer;

FIG. 14 is a perspective view showing a transverse cross-section view showing a major construction of a variant of a support section of the capsule endoscope on the distal end portion of the introducer of the endoscope system of the third embodiment;

FIG. 15 is a longitudinal cross-section view showing a major portion of a suturing machine of an endoscope system according to a fourth embodiment of the present invention;

FIG. 16A is a perspective view showing a state in which a living body tissue portion is sucked on a side opening of a movable hood of the endoscope system of the fourth embodiment;

FIG. 16B is a perspective view showing a state in which the hood is moved in its side-opening closed direction and the living body tissue is penetrated by a needle;

FIG. 17 is a perspective view showing a state just before a second T bar is released after being penetrated in a region other than a detained first T bar region at a suturing operation by a suturing machine of the endoscope system of the fourth embodiment;

FIG. 18 is an oblique view showing a state in which, after the second T bar has been detained at the time of a suturing operation by the suturing machine of the endoscope system of the fourth embodiment, these T bars are narrowed therebetween and tightened up by clipping tool;

FIG. 19 is a perspective view showing a state of suturing together two separate parts of the living body tissue by clipping together the T bars at the time of the suturing operation by the suturing machine of the endoscope system of the fourth embodiment;

FIG. 20 is a perspective view showing an operation state of a tightening tool at the time of the suturing operation by the suturing machine of the endoscope system of the fourth embodiment;

FIG. 21 is a perspective view showing a state in which the suturing machine of the endoscope system of the fourth embodiment is inserted from the esophagus into the stomach beyond a wire guide;

FIG. 22 is a perspective view showing a state in which the suturing machine is operated while securing a visual field with a plurality of capsules of the endoscope system of the fourth embodiment;

FIG. 23A is a perspective view showing a state in which an EMR is done while observing an endoscope with a plurality of capsule endoscopes of the endoscope system of a fifth embodiment of the present invention;

FIG. 23B is a plan view showing a distal end face of the insertion section of the endoscope;

FIG. 24 is a longitudinal cross-sectional view showing an inner structure of the capsule endoscope of the endoscope system of the fifth embodiment;

FIG. 25 is a perspective view showing a state of observing an endoscope with a first capsule endoscope having a wide visual field angle type observation optical system and a second capsule endoscope having an enlarged visual field type observation optical system;

FIG. 26 is an explanatory view showing a state in which a capsule endoscope of an endoscope system of a sixth embodiment of the present invention is coupled to a distal end of an endoscope;

FIG. 27 is an explanatory view for explaining a state in which the capsule endoscope of the endoscope system of the sixth embodiment is fixed to the living body tissue of a human body;

FIG. 28 is an explanatory view showing a state of use of the endoscope system of the sixth embodiment;

FIG. 29 is a diagrammatic view showing an endoscope system of a seventh embodiment of the present invention;

FIG. 30 is a diagrammatic view showing an endoscope system of an eighth embodiment of the present invention;

FIG. 31 is a diagrammatic view showing an endoscope system of a ninth embodiment of the present invention;

FIG. 32 is a diagrammatic view showing a method for inserting a capsule endoscope into a body cavity of a human subject which is operated by an endoscope system of a tenth embodiment;

FIG. 33 is an explanatory view for explaining a fixing operation of the endoscope capsule by the endoscope system of the tenth embodiment of the present invention;

FIG. 34 is a diagrammatic view showing a method for inserting an endoscope capsule into a body cavity of a human subject by an endoscope system of an eleventh embodiment of the present invention;

FIG. 35 is a diagrammatic view showing a state in which an endoscope capsule is inserted into a body cavity of a human subject by an endoscope system of a twelfth embodiment; and

FIG. 36 is an explanatory view for explaining the fixing operation of the capsule endoscope into a body cavity of a human subject by the endoscope system of the twelfth embodiment

### DETAILED DESCRIPTION OF THE INVENTION

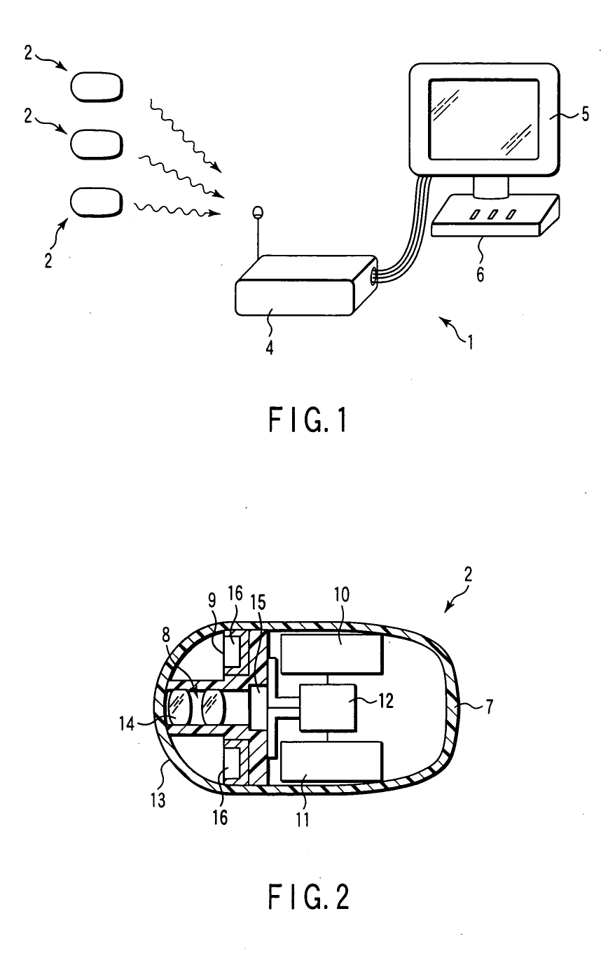

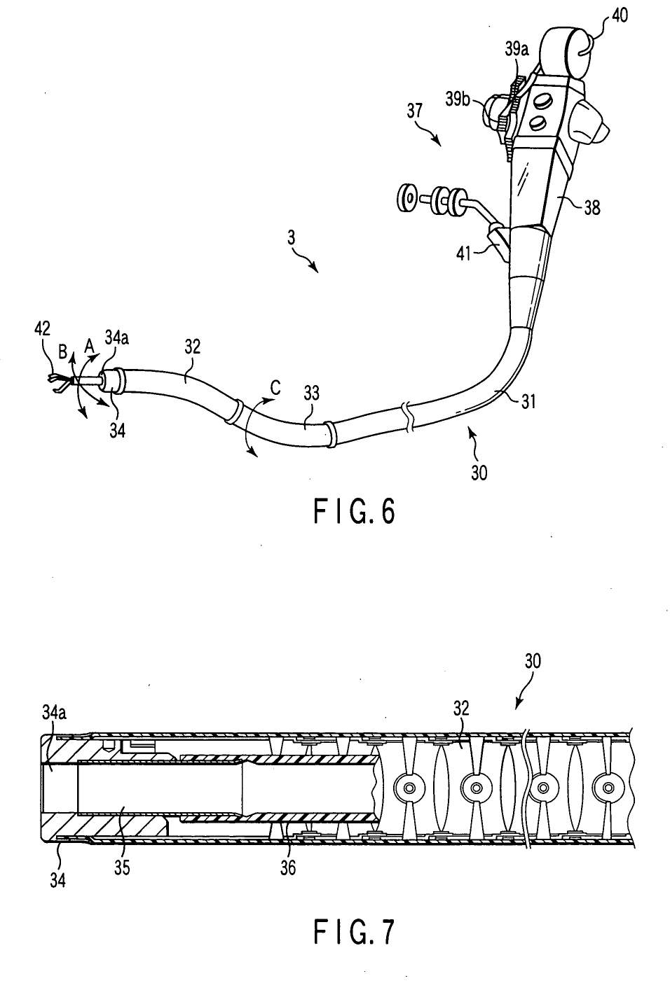

Preferred embodiments of the present invention will be described, with reference to the accompanying drawings. Observation devices are used in the embodiments. The devices can be retained in the subject and transmit, by radio, the data representing an interior image of the subject to a receiving device provided outside the subject. Hereinafter they will be referred to as “capsule endoscopes”. Nonetheless, they are not limited to devices shaped like a capsule. Observation devices of such shapes shown in the drawings shall be called “capsule endoscopes”. FIGS. 1 to 9 show a first embodiment of the invention. FIG. 1 is a diagrammatic view generally showing an endoscopic system 1 of the first embodiment. The endoscope system includes a system having a plurality of capsule endoscopes (observation devices) 2 and an introducer 3 comprised of a treating device as shown in FIG. 6.

The system for the capsule endoscopes 2 has a receiver 4, a monitor 5 and a console (or a keyboard) 6. FIG. 2 shows an inner structure of the capsule endoscope 2. The capsule endoscope 2 has a capsule type casing 7. Within the casing 7 an observation optical system 8, an illumination optical system 9, an antenna 10, a cell unit 11 and a control circuit 12 are provided.

A transparent hood 13 is provided at a forward end of the casing 7. On the inner surface of the transparent hood, the observation optical system 8 is provided at a central area. The illumination optical system 9 is arranged around the observation optical system 8. The observation optical system 8 has an object lens 14 and an image pickup element 5 arranged at an imaging position of the object lens 14.

The control circuit 12 has a built-in camera control unit (CCU), etc., connected to the image pickup element 15. The antenna 10 and cell unit 11 are connected to the control circuit 12. The illumination optical system 9 has a plurality of LEDs 16. Each LED 16 is connected to the control circuit 12.

At the operation time of the capsule endoscope 2, illumination light exit from the LEDs 16 in the illumination optical system 9. An observation image is imaged by the object lens 14 of the observation optical system and is taken by the image pickup element 15. At this time, the observation image is converted by the image pickup element 15 to an electric signal. The electric signal of the image pickup element 15 is input to the control circuit 12 and then it is output as a radio wave from the antenna 10.

The wave signal which is output from the capsule endoscope 2 is received by the receiver 4 and is sent to the monitor 5 connected to the receiver. And the observation signal observed by the observation optical system 8 is displayed on the monitor 5.

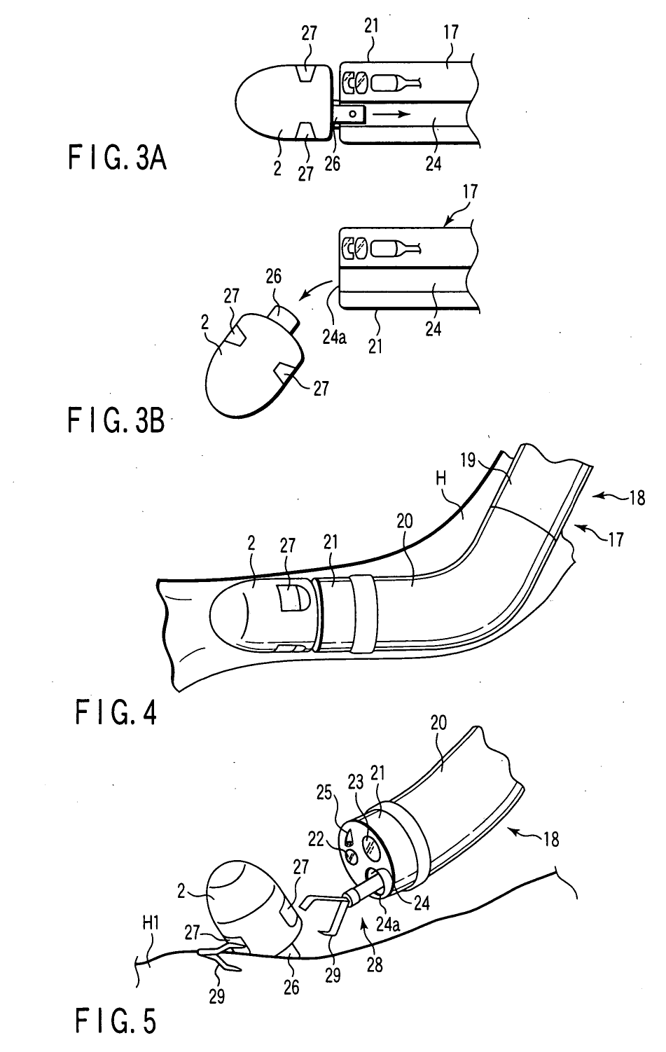

FIGS. 3A to 5 are views for explaining one practical method for detaining the capsule endoscope 2 of the endoscope system 1 of the present embodiment on a body cavity of a human subject. According to the present embodiment, a flexible endoscope 17 is used as a treating tool for transporting the capsule endoscope 2. The endoscope 17 has an elongated insertion section 18 inserted into the body cavity of the human subject. As shown in FIG. 4, a curved section 20 is connected to a distal end of an elongated flexible section 19 in the insertion section 18 and a rigid forward end section 21 is connected to a distal end of the curved section 20. As shown in FIG. 5, an observation window 22, an illumination window 23, a distal window opening section 24a of a treating tool insertion channel 24 and a water/air supply nozzle 25 are provided at the distal end face of the rigid distal end section 21. A suction tube, not shown, is connected to the treating tool insertion channel 24.

As shown in FIG. 3A, a rearwardly extending connection projection 26 is provided at a base end of the endoscope 2. A plurality of recesses 27 for fixing are provided, along a peripheral direction, at an outer peripheral surface of the basic end portion of the capsule endoscope 2.

Where the capsule endoscope 2 is to be transported, the projection 26 of the capsule endoscope 2 is inserted into a distal end opening 24a of the treating tool insertion channel 24 of the endoscope 17 as shown in FIG. 3A and, thus, it is set there. In this state, a suction force is exerted on the treating tool insertion channel 24 of the endoscope 17. By doing so, the capsule endoscope 2 is connected to the distal end of the insertion section 18 of the endoscope 17, so that the endoscope 2 is fixed there.

In this state, as shown in FIG. 4, the insertion section 18 of the endoscope 17 is inserted into a body cavity H of a patient. At this time, the insertion operation of the insertion section 18 of the endoscope 17 is done while observing an observation image of the capsule endoscope 2. And the capsule endoscope 2 is brought to a target region in the body H of the patient. It is to be noted that the patient may swallow the capsule endoscope 2 itself down into the body cavity of the patient.

At a point of time at which the capsule endoscope reaches the target region in the body cavity of the patient, the inside of the treating tool insertion channel 24 of the endoscope 17 is switched to an air supply state. By doing so, as shown in FIG. 3B, the capsule endoscope 2 is dropped down from the distal end opening 24a of the treating tool insertion channel 24 of the endoscope 17.

Then, as shown in FIG. 5, a pair of chip forceps 28 is inserted past the treating tool insertion channel 24 of the endoscope 17 into the body cavity H of the patient. By the chip forceps 28, the capsule endoscope 2 is fixed to, for example, a mucosa H1 of a target region (a region to be detained) of the body cavity of the patient. FIG. 5 shows the state in which a pair of clip fixing device 28 is extended from the distal end of the treating tool insertion channel 24 of the endoscope 17 and the capsule endoscope 2 is fixed in place in the body cavity by means of clips. At this time, the clips 29 of the clip fixing device 28 are fixed in place in such a state as to anchor their claws to the recess 27 of the capsule endoscope 2 on one hand and to the mucosa H1 of the body cavity H on the other hand.

FIG. 6 shows an outer appearance of an introducer 3 which is used in combination with the capsule endoscope 2. The introducer 3 has, like the endoscope 17, an elongated insertion section 30 which is inserted into the body cavity H. The insertion section 30 includes a so-called two-stepped curved structure at the elongated flexible tube section 31. The curved structure has first and second curved sections 32 and 33 that can be operated individually.

FIG. 7 is a cross-sectional view showing a distal end portion of the insertion section 30 of the introducer 3. As shown in FIG. 7, a cylindrical distal end member 34 is coupled to the distal end portion of the first curved section 32 and a distal end portion of a coupling tube 35 is fitted in, and fixed to, a channel opening 34a of the distal end member 34. The base end portion of the coupling tube 35 is extended backwardly of the distal end member 34. The distal end portion of a channel tube 36 is coupled to the backwardly extending portion of the coupling tube 35.

An operation section 37 is coupled to a proximal end of the insertion section 30. At the outer peripheral surface of the rear portion of a grip section 38 on the operation section 37 which is grasped by the operator, a first knob 39a and a second knob 39b are provided. The first knob 39a is so designed as to bend the first curved section 32, for example, in an up/down direction as indicated by an arrow A in FIG. 6 and the second knob 39b is so designed as to bend the first curved section 32 in a right/left direction as indicated by an arrow B in FIG. 6. Further, at the base rear end portion of the grip section 38, a third knob 40 is provided so as to allow the second curved section to be bent in two directions as indicated by an arrow C in FIG. 6.

A treating tool insertion section 41 projects from the forward end portion of the grip section 38. The proximal end of the channel tube 36 is coupled to the treating tool insertion section 41. The treating tool 42 is inserted from the treating tool insertion section 41 into the channel tube 36 and further from the channel tube 36 past the coupling tube 35. Thus, the tool 42 can be forwardly extended via the channel bore 34a of the distal end member 34. As can be understood from the above, the introducer 3 is identical in structure to the insertion section 18 of an ordinary flexible endoscope 17, except for two respects. First, it has no observation optical system. Second, the distal end of the insertion section has an opening, through which the channel tube 36, i.e., the treating tool insertion channel, can be guided.

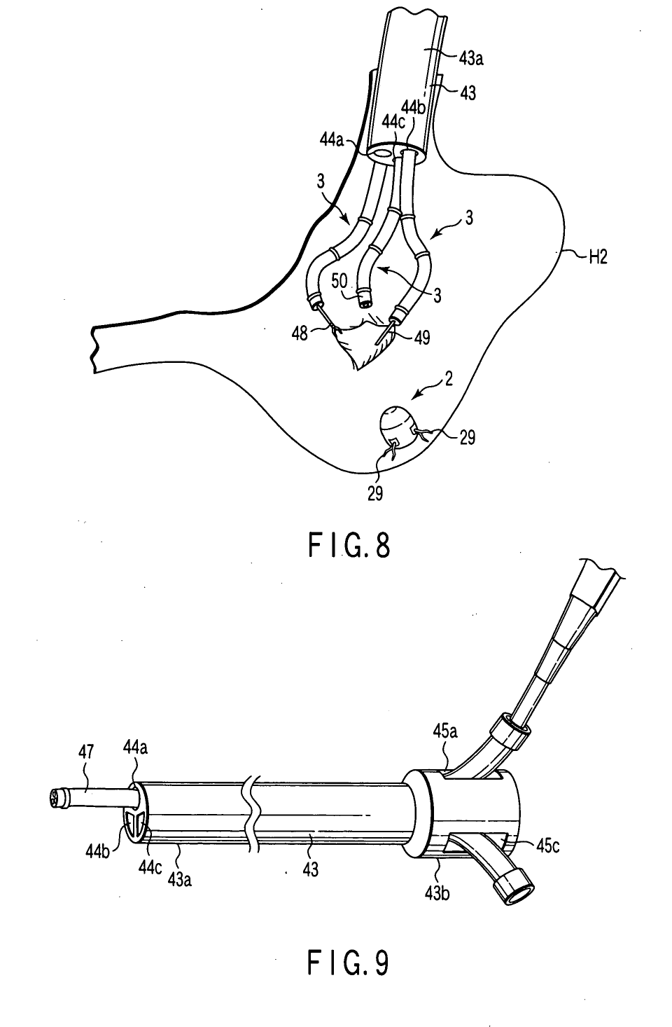

Further, the introducer 3 of the present embodiment is further used in combination with a three-lumen overtube of a treating tool for guide use as shown in FIG. 8. The three-lumen overtube 43 has an elongated overtube body 43a as shown in FIG. 9. A proximal-side end section 43b is provided on the base end of the overtube body 43a.

Three lumens 44a, 44b and 44c are provided at the inside of the overtube body 43a and extend as a parallel array along an axial direction. At the outer peripheral surface of the proximal-side end section 43b, three treating tool introducing inlets 45a, 45b and 45c are provided in a manner to communicate with the three lumens 44a, 44b and 44c, the two treating tool introducing inlets 45a and 45c only being shown in FIG. 9. At the time of using the three lumen overtube 43, three independent treating tools are inserted via the corresponding three treating tool introducing inlets 45a, 45b and 45c into the three lumens 44a, 44b and 44c. For example, a narrow-diameter scope 47 for guide use is inserted into the first treating tool introducing inlet 45a as shown in FIG. 9. And the three-lumen overtube 43 is inserted into the body cavity H of the patient with the narrow-diameter scope 47, that is, a scope initially inserted into the body cavity H, as a guide.

Now an operation of the above structure will be explained below. FIG. 8 shows a state in which the endoscope system 1 of the present invention is used. Here, as set out above, the capsule endoscope 2 is initially inserted by the transport-use endoscope 17 into a target region in the stomach H2 of the patient. After this, the capsule endoscope 2 is clipped by the clips 29 of the clip fixing device 28 to the recess 27 of the capsule endoscope 2 on one hand and to the mucosa H1 of the body cavity H on the other hand and detained at a target region in the stomach H2 of the patient and held in place.

In this state, the digital end portion of the narrow-diameter scope 47 is inserted from the mouth past the esophagus into the stomach H. Then, the three-lumen overtube 43 is inserted into the body cavity H with the narrow-diameter scope 47 as a guide. At this time, the insertion operation of the three-lumen overtube 43 is observed by the capsule endoscope 2. And the distal end portions of the three-lumen overtube 43 is retained in a state in which it is inserted from the esophagus of the patient to the near-cardiac part of the stomach.

Then, into the three lumens 44a, 44b and 44c of the three-lumen overtube 43 corresponding introducers 3 are introduced. And into three introducers 3 independent treating tools, for example, grasping forceps 48, electrosurgical knife 49 and a water supply tube 50 are inserted as shown in FIG. 8. At this time, a necessary procedure, such as the removal of the mucosa, is performed while observing the operation of the introducer 3 with the capsule endoscope 2 from the inner side of the stomach H2. The capsule endoscope 2 allows a region of interest to be observed in an enlarged fashion by a zooming operation (close view) of the observation optical system 8 from the outside or a whole operation to be grasped by a wide angle visual field and, by doing so (distant view), a surgical procedure to be easily done.

The above-mentioned structure ensures the following advantages. That is, in the endoscope system 1, the observation optical system 8 of the capsule endoscope 2 allows a surgical procedure to be done at a target region in the body cavity of the patient and, while observing the operation of the introducer 3, to be done at a region of interest in the body cavity of the patient by those independent treating tools inserted into the introducers, for example, the grasping forceps 48, electrosurgical knife 49 and water supply tube 50 as shown in FIG. 8. The observation optical system 8 can therefore be dispensed with in the insertion section 30 of the introducer 3 that is used as treating device. This reduces the size of the insertion section 30 of the introducer 3. Further, even if the first and second curved section 32 and 33 are so operated as to be bent independently and the shape of the insertion section 30 of the introducer 3 varies in a complicated fashion, it is possible to grasp the state of the insertion section 30 of the introducer 3 positively under the observation optical system 8 of the capsule endoscope 2.

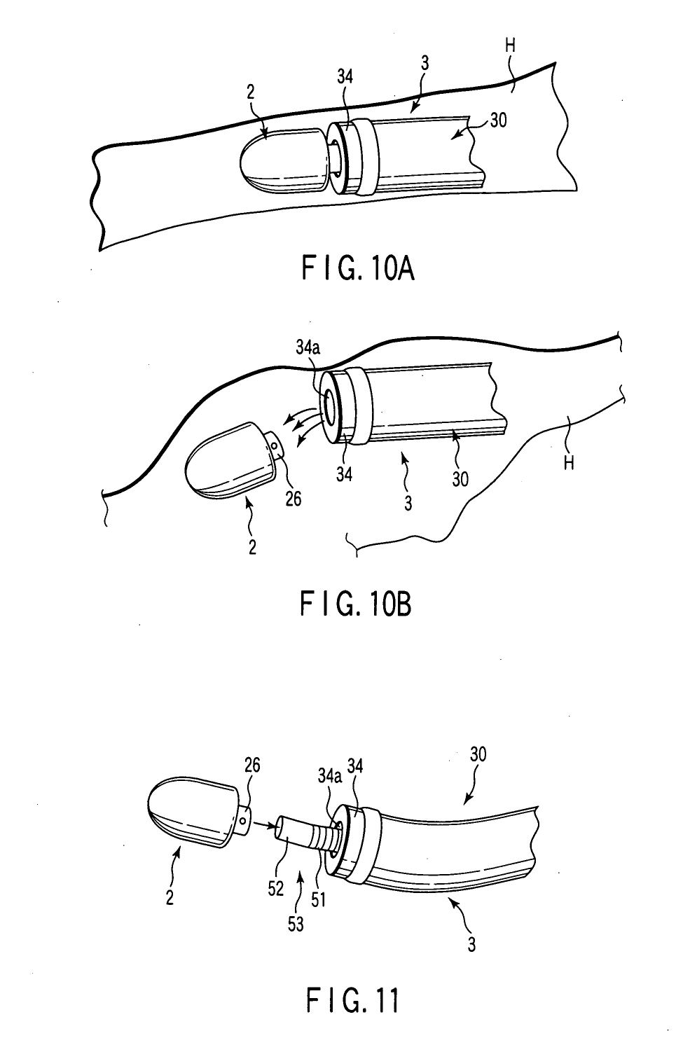

FIGS. 10A, 10B and 11 show a second embodiment of the present invention. The second embodiment is different from the first embodiment (see FIGS. 1 to 9) in that a different method is adopted in detaining the capsule endoscope 2 of the endoscope system as will be set out below.

That is, by the use of an introducer 3 of the endoscope system 1 as shown in FIG. 10A, it is possible to introduce a capsule endoscope into a body cavity, detain it there and recover it from the body cavity. Here, as shown in FIG. 10A, a projection 26 of the capsule endoscope 2 is so set as to be inserted in a channel bore 34a of a distal end member 34 of the introducer 3. In this state, a suction force is exerted on a channel tube 36 of the introducer 3. By doing so, the capsule endoscope 2 is coupled to the distal end member 34 of the introducer 3 and fixed to it.

In this state, as shown in FIG. 10A, an insertion section 30 of the introducer 3 is inserted into a body cavity H of a patient. At this time, the insertion operation of the insertion section 30 of the introducer 3 is performed while observing an observation image of the capsule endoscope 2, and the capsule endoscope 2 is guided onto a target region in the body cavity H of the patient.

When the capsule endoscope reaches the target region in the body cavity of the patient, the air is supplied into the channel tube 36 of the introducer 3. By doing so, as shown in FIG. 10B, the capsule endoscope 2 is dropped down from the channel bore 34a of the distal end of the distal end member 34 of the introducer 3.

FIG. 11 shows the operation of recovering the capsule endoscope 2. Here, use is made of forceps 53 with a magnet 52 attached to the distal end of an elongated insertion section 51 which is inserted into a channel bore 34 of the introducer 3. By forwardly projecting the magnet 52 on the forward end of the forceps 53 from the channel bore 34a of the introducer 3 and magnetically attracting the projection 26 of the capsule endoscope 2 with a magnetic force of the magnet 52 on the forwarding end of the forceps 53, the capsule endoscope 2 is recovered from the body cavity H of the patient. It is to be noted that the projection 26 of the capsule endoscope 2 is made of a magnetic material which can be magnetically attracted with a magnetic force of the magnet 52 on the forward end of the forceps 53.

According to the present embodiment, the capsule endoscope 2 and introducer 3 can be aligned in series array in an axial direction of the introducer. Since any observation optical system 8 can be omitted at the insertion section 30 of the introducer 3 as in the case of the first embodiment, the diameter of the insertion section 30 of the introducer 3 can be made narrower.

By dropping the capsule endoscope 2 from the channel bore 34a of the distal end of the distal end member 34 of the introducer 3 it is possible to detain the capsule endoscope 2 in a fixed state in the body cavity H of the patient. By doing so, the first and second curved sections 32 and 33 of the introducer 3 are independently bend-operated and, even if the shape of the insertion section 30 of the introducer 3 varies in a complicated fashion, it is possible to grasp the state of the insertion section 30 of the introducer 3 positively with an observation optical system 8 of the capsule endoscope 2.

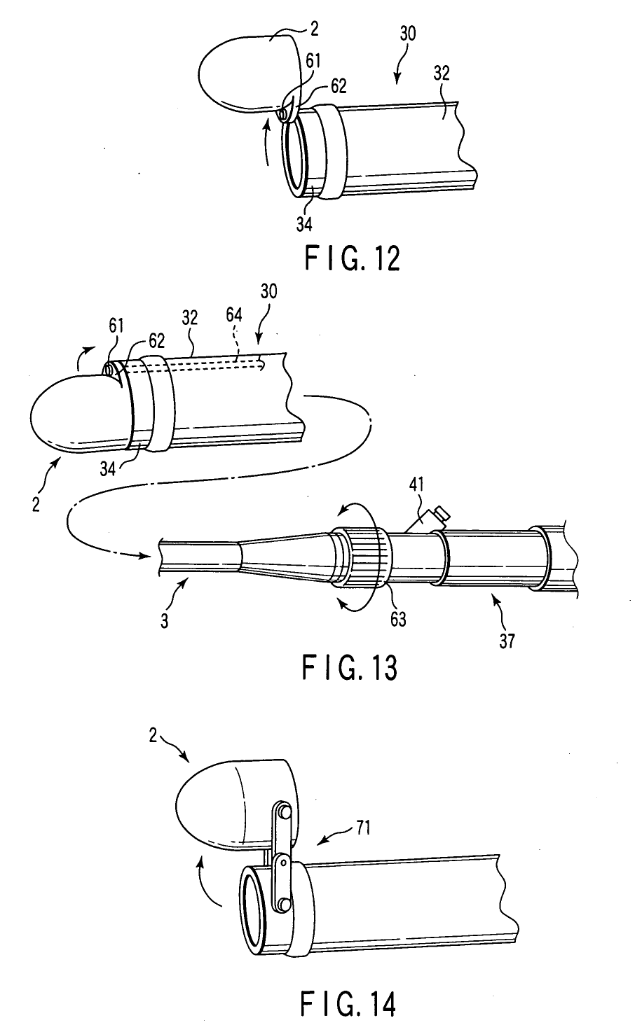

FIGS. 12 and 13 show a third embodiment of the present invention. The third embodiment is different from the first embodiment in terms of the support mechanism of the capsule endoscope 2 in the endoscope system 1 (see FIGS. 1 to 9) as will be set out below.

That is, in the third embodiment, a support shaft (endoscope support section) 61 of a capsule endoscope 2 is rotatably supported at the marginal edge portion of a distal end of a distal end member 34 of an introducer 3. A shaft fixing section 62 is provided at an outer peripheral edge portion of a basic end of the capsule endoscope 2 and the support shaft 61 is coupled, in a fixed state, to the shaft fixing section 62.

A rotation operation knob (endoscope operation section) 63 is provided on an operation section 37 of an introducer 3 as shown in FIG. 13. To the operation knob 63, a proximal end of a torque transmission mechanism such as a torque wire 64 is coupled. The distal end of the torque wire 64 is fixed to the support shaft 61 of the capsule endoscope 2.

With the rotation operation of the rotation operation knob 63, the capsule endoscope 2 is rotated, by the torque wire 64, about the support shaft 61. By doing so, the capsule endoscope is so supported as to allow the capsule endoscope to be coupled to a position in which, as shown in FIG. 13, the capsule endoscope 2 closes the channel bore 34a of the distal end member 34 of the introducer 3 and allow the capsule endoscope 2 to be moved to a position in which, as shown in FIG. 12, the channel bore 34a of the distal end member 34 of the introducer 3 is opened.

Even in this embodiment, the capsule endoscope 2 and introducer 3 can be aligned in series array in the axial direction. For this reason, an observation optical system 8 can be omitted at the insertion section 30 of the introducer 3 as in the case of the first embodiment and the diameter of the insertion section 30 of the introducer 3 can be made narrower.

FIG. 14 shows a variant of the support mechanism of the capsule endoscope at a distal end of the introducer 3 of the endoscope system 1 of the third embodiment (see FIGS. 12 and 13). In this variant, a link mechanism 71 is provided at the marginal edge portion of a channel bore 34a of a distal end member 34 of the introducer 3 to allow the capsule endoscope 2 to be moved to a lateral position. The link mechanism 71 supports the capsule endoscope 2 to allow the capsule endoscope 2 to be coupled to a position in which the capsule endoscope 2 closes the channel bore 34a of the distal end member 34 of the introducer 3—see FIG. 13—and allow the capsule endoscope 2 to be eccentrically moved to a lateral position in which the channel bore 34a of the distal end member 34 of the introducer 3 is opened as shown in FIG. 14.

Even in this variant it is possible to obtain the same advantage as that of the third embodiment.

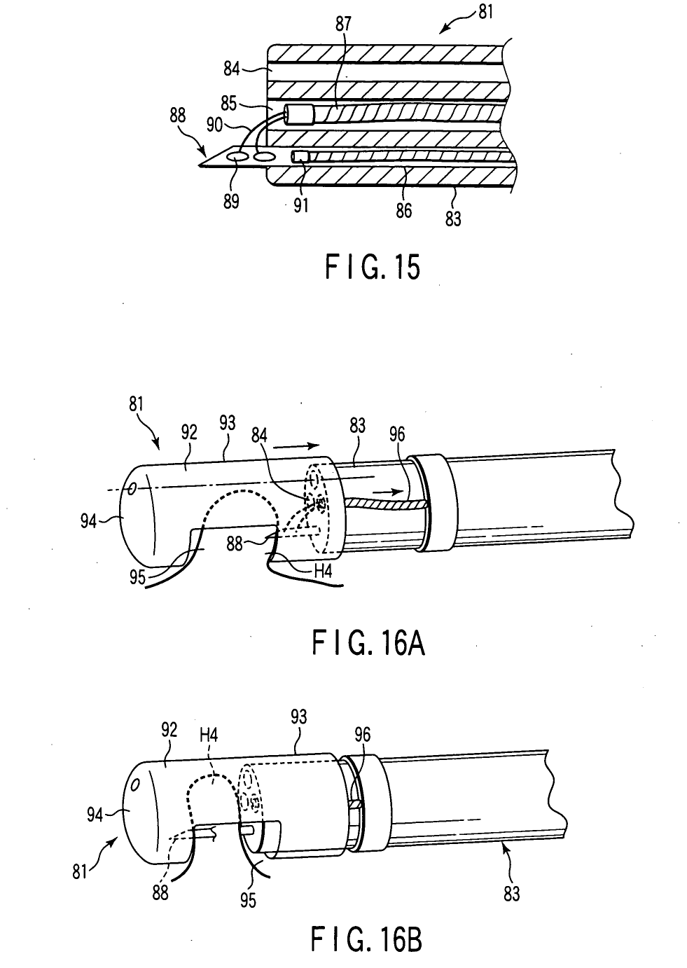

FIGS. 15 to 22 show a fourth embodiment of the present invention. The fourth embodiment is such that, in place of the introducer 3 of the first embodiment (see FIGS. 1 to 9), a suturing machine 81 as shown in FIG. 15 is provided as a treating device of the endoscope system 1 which is introduced via the mouth into the body cavity. The fourth embodiment provides a system comprising this suturing machine 81 and a plurality of capsule endoscopes 82.

FIG. 15 is a cross-sectional view showing a distal end portion of the suturing machine of the fourth embodiment. The suturing machine 81 has an elongated shaft body having three lumens 84, 85 and 86. These lumens 84, 85 and 86 are used, respectively, as a suction bore 84, a clipping tool insertion bore 85 and a needle insertion bore 86. Here, the clipping tool 87 is set in the clipping tool insertion bore 85 and the needle 88 is set in the needle insertion bore 86. The needle 88 is of a hollow type and has two bars 89a, 89b, thread 90 and a pusher 91.

A movable type hood 92 is provided at the distal end of the suture machine 81 as shown in FIG. 16A and has a cylindrical hood body 93. A forward end face is formed as a closed end 94 and a side opening 95 is formed in the outer peripheral portion of the hood body 93 at one side surface.

In a shaft body 83 of the suture machine 81, a hood operation wire 96 for allowing the movable type hood 92 to be slidably moved along an axial direction is so inserted as to be movable back and forth along the axial direction. The hood operation wire 96 is moved back and forth in the axial direction by operating an operation section, not shown, coupled to the proximal side of the shaft 83 of the suturing machine 81.

The operation of the fourth embodiment will be explained below. At the time of using the endoscope system 1 of the fourth embodiment, a plurality of capsule endoscopes 82 is initially detained in the body cavity H of a patient, for example, at target region in the stomach H2 as shown in FIG. 22.

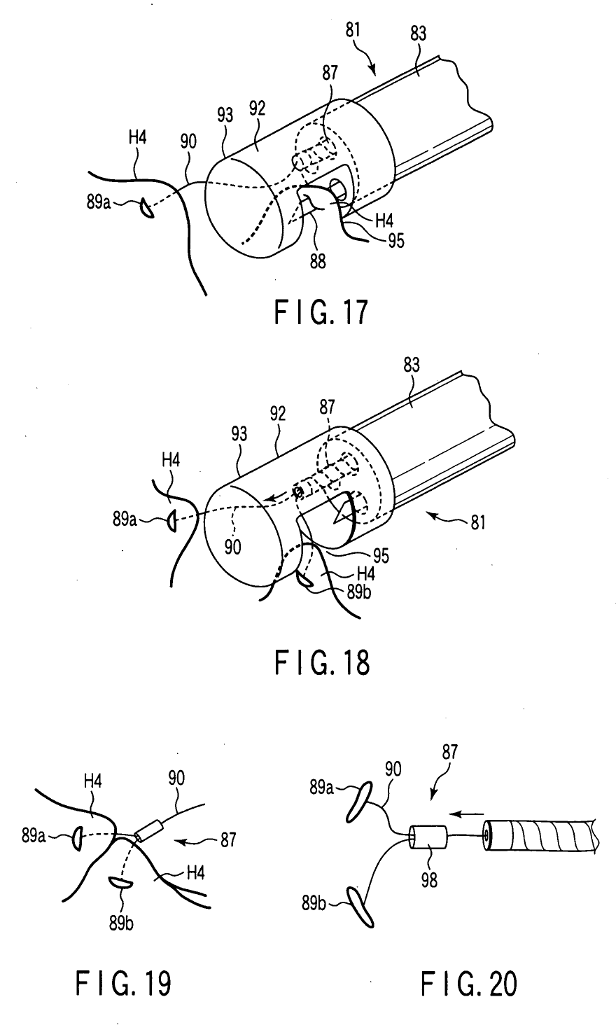

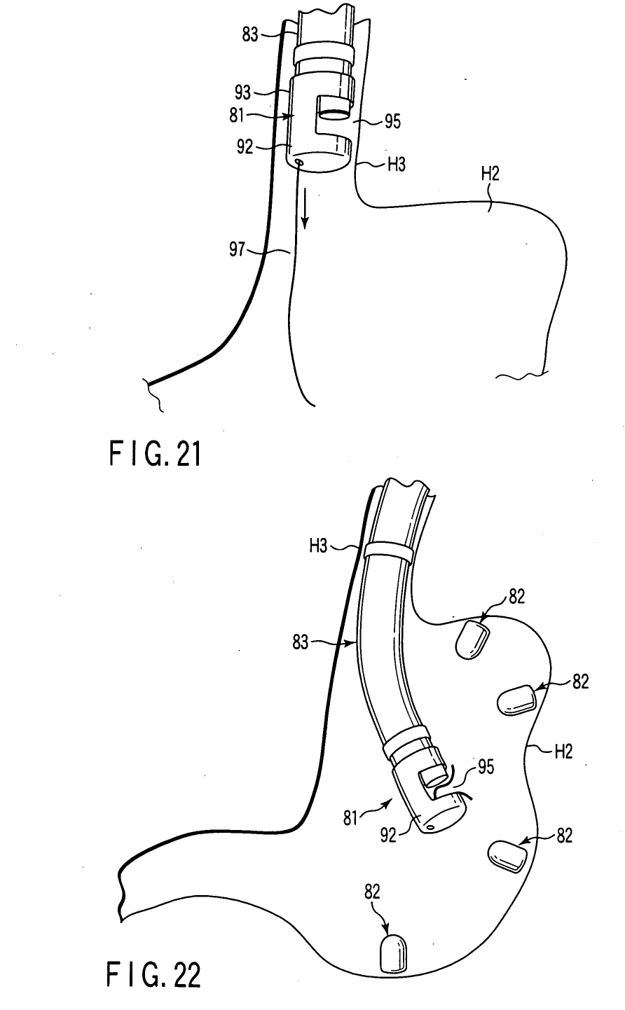

Then the suturing machine 81 is inserted as shown in FIG. 21 along a guide wire 97 into the stomach H2 past the esophagus H3. In this state, as shown in FIG. 22, while securing a visual field with the plurality of capsule endoscopes 82, the suturing machine 81 is operated and a piercing/suture is done by the suturing machine 81. FIGS. 17 to 19 show explanatory views for explaining the piercing/suture procedure by the suturing machine 81.

At a time of operating the suturing machine 81, as shown in FIG. 16A, the movable hood 92 is so set as to be pushed ahead of the suturing machine 81. In this state, a suction force is applied to the side opening 95 of the hood 92. By doing so, a living body tissue H4 such as the mucosa is sucked into the side opening 95 of the hood 92 with the suction force applied.

In this state, a pull operation is performed by pulling the hood 92 toward the proximal side. The movable hood 92 is thereby moved to close the side opening 95 as is illustrated in FIG. 16B. At this time, the living body tissue H4 can be pierced with the needle 88 and, in this state, the first T bar 89a is pushed out of the needle 88. By doing so, the first T bar 89a is detained on the living body tissue H4.

Then, as shown in FIG. 17, at an area separate from the first T bar 89a area detained by the suturing operation by the suturing machine 81, the needle 88 is penetrated and a second T bar 89b is released. FIG. 17 shows a state just before the releasing of the second T bar 89b.

FIG. 18 shows a state in which, after detaining the second T bar 89b on an area separate from the first T bar 89a area by the suturing operation by the suturing machine, the T bars 89a and 89b are narrowed together by the clipping tool 87 for clipping.

FIG. 19 shows a state in which, when clipping these T bars 89a, 89b by the suturing operation by the suturing machine 81, the two separating parts of the living body tissue H4 are pulled together for the suture to be done there.

FIG. 20 shows an operation state of the clipping tool 87 at a time of the suture operation by the suturing machine 81. Here, the clipping tool 87 is comprised of a tightened member 98 made of an elastic body such as a rubber and, by pushing out the thread 90 in a squeezing-up direction and tightening it up, the two separate parts of the living body tissue H4 are pulled closer to each other and sutured together.

In this structure, since the suturing machine 81 can be observed with the plurality of capsule endoscopes 82, it is possible to omit the observation optical system 8 in the shaft body 83 of the suturing machine 81 and, for this reason, the size of the shaft body 83 of the suturing machine 81 can be narrowed. Even if the shape of the shaft body 83 of the suturing machine 81 varies in a complicated fashion, it is possible to positively grasp the state of the shaft body 83 of the suturing machine 81.

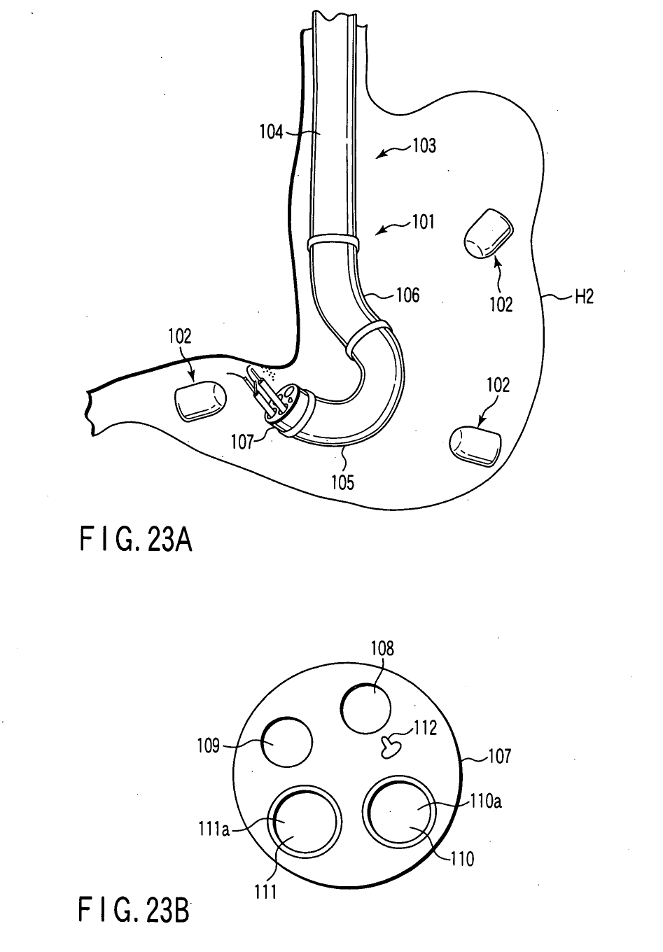

FIGS. 23A to 25 show a fifth embodiment of the present invention. The fifth embodiment is different from the first embodiment (see FIGS. 1 to 9) in terms of the structure of the endoscope system 1 as will be set out below.

That is, the fifth embodiment comprises, as shown in FIG. 23A, a flexible endoscope 101 in combination with a plurality of capsule endoscopes 102. The endoscope 101 has an elongated insertion section 103 to be inserted into the body cavity H of a patient. On the insertion section 103, a so-called two-stepped structure is used which has two (first and second) curved sections 105 and 106 on the distal end side of an elongated flexible tube section 104, these curved sections being individually operable.

Further, a rigid distal end section 107 is coupled to the forward end of the first curved section 105. As shown in FIG. 23B, an observation window 108, an illumination window 109, distal end openings 110a and 111a of treating tool insertion channels 110 and 111, and an air/water supply nozzle 112 are provided at the distal end face of the rigid distal end section 107.

FIG. 23A shows a state in which an EMR (Endoscopic Mucosal Resection) is performed in the stomach H2 of the patient while observing the insertion section 103 of the endoscope 101 with a plurality of capsule endoscopes 102.

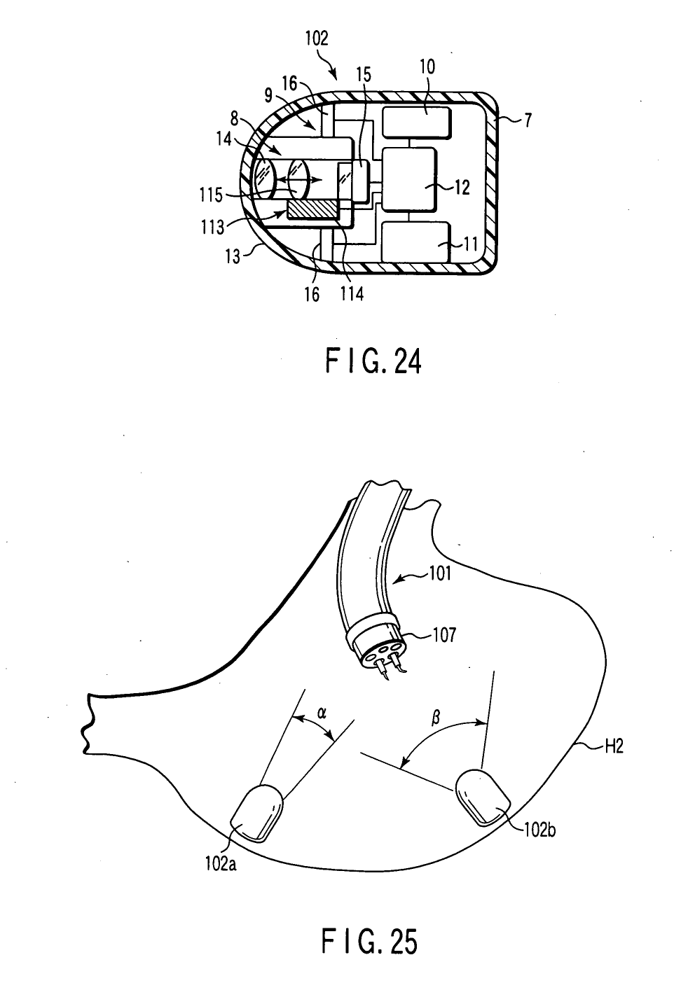

FIG. 24 shows an inner structure of the capsule endoscope 102. The main portion of the capsule endoscope 102 is the same as that of the capsule endoscope 2 of the first embodiment. In the fifth embodiment, the same reference numerals are employed to designate parts or elements corresponding to those shown in the capsule endoscope 2 of the first embodiment and their explanations are omitted for brevity sake. The fifth embodiment is different from the first embodiment particularly in terms of providing a zooming function section 113 in the observation optical system 8. The zooming function section 113 has a piezoelectric actuator 114 and a zoom lens 115 configured to be moved by the piezoelectric actuator 114 along the optical axis direction of the observation optical system 8.

In the capsule endoscope 102 of the present invention, for example, a radio wave signal is received from an external source, not shown, by an antenna 10, a control signal is output via a control circuit 12 to the piezoelectric actuator 114 and the zoom lens 115 is moved along the optical axis direction of the observation optical system 8. By doing so (close view or distant view), it is possible to adjust the optical performance (an angle of view, focal distance, focal depth, etc.).

Further, FIG. 25 is a view showing a state in which two capsule endoscopes 102a and 102b of different optical performances are detained in the stomach H2 of the patient. Here, the capsule endoscope 102a allows the zoom lens 115 to be driven by the piezoelectric actuator 114 and, by doing so, it allows an observation to be made as an enlarged view (close view) at a visual field angle a narrowed. Further, the second capsule endoscope 102b allows the zoom lens 115 to be driven by a piezoelectric actuator 114 and it allows an observation to be made as a wide angle image (distant view) at a visual field angle β broadened. In the Figure, it is seen that an EMR is done in the stomach H2 of the patient while observing the insertion section 103 of the endoscope 101 with these two capsule endoscopes 102a and 102b.

It is to be noted that the capsule endoscope may be of such a type as to have an initially set optical function (has no zooming function). In this case, the capsule endoscope is simpler in structure and lower in cost involved.

Since, in the above-mentioned structure, the endoscope 101 can be observed with a plurality of capsule endoscopes 102a, 102b, it is possible to omit the observation optical system 8 of the endoscope 101 as in the case of the first embodiment, so that the size of the endoscope 101 can be made narrower. Even if, therefore, the shape of the endoscope 101 varies in a complicated fashion, it is possible to positively grasp the state of the endoscope 101 with the observation optical systems 8 of the capsule endoscopes 82.

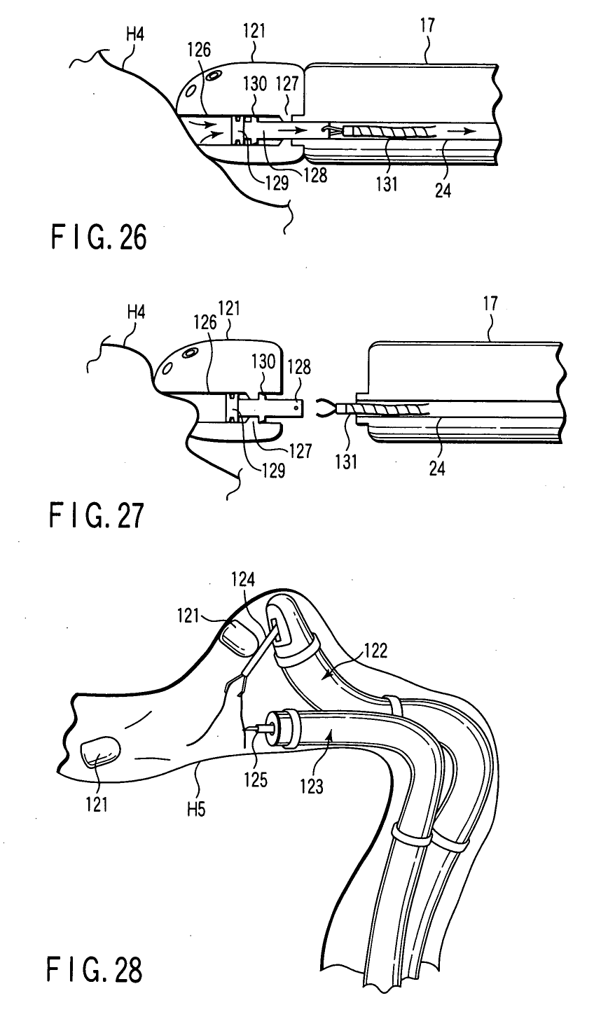

FIGS. 26 to 28 show the sixth embodiment of the present invention. The sixth embodiment is different from the first embodiment (FIGS. 1 to 9) in terms of the structure of the endoscope system 1 as will be set out below.

That is, in the sixth embodiment, a plurality of capsule endoscopes 121 inserted via the anus into the large intestine H5 of the patient H5 and detained there and a surgical operation can be done by introducing a plurality of introducers (side-viewing introducer 122 and direct-viewing introducer 123) into the large intestine H5. In this case, grasping forceps 124 is inserted into the side-viewing introducer 122 and a direct-viewing introducer 123 is inserted into the direct-viewing introducer 123.

In the sixth embodiment, as shown in FIG. 28, with the inner wall of the large intestine H5 pushed up by the grasping forceps 124 of the side-viewing introducer 122, the living body tissue, such as the inner wall part of the large intestine wall H5 can be removed by the electrosurgical knife 125 of the direct-viewing introducer 123.

FIGS. 26 and 27 are an explanatory view for explaining the operation of temporarily fixing the capsule endoscope 121 of the endoscope system 1 of the sixth embodiment to the living tissue, etc., of the patient. Here, a suction bore 126 is extended along the axial direction of the capsule endoscope 121. An engaging projection 127 is provided on the inner wall surface of a base end portion of the suction hole 126.

In the suction bore 126, a piston-like movable member 128 is so provided as to be movable back and forth along the axial direction of the suction bore 126. A sealing member 129 is fixed to the forward end of the movable member 128. On the intermediate way of the movable member 128 an engaging portion 130 is formed to allow it to engage with and disengage from the engaging projection 127 of the engaging bore 126.

In the sixth embodiment, as the treating tool for transporting the capsule endoscope 121 use is made of a flexible endoscope 17 as in the case of the first embodiment. Into a treating tool insertion channel 24 of the endoscope 17, the grasping forceps 131 is inserted into the treating tool insertion channel 24 of the endoscope 17. As seen from FIG. 26, the capsule endoscope 121 is fixed to the endoscope 17 in a way to be grasped by the grasping forceps 131.

At a time of temporarily fixing the capsule endoscope 121 to the living body tissue H4, etc., the movable member 128 is externally pulled by the grasping forceps 131 with the suction bore 126 of the capsule endoscope 121 pushed against the living tissue H4. By doing so, as shown in FIG. 27, a negative pressure acts in the suction bore 126 of the capsule endoscope 121 and, with the living body tissue H4 sucked into the suction bore 126, the capsule endoscope 121 is temporarily fixed to the living tissue, etc., of the patient.

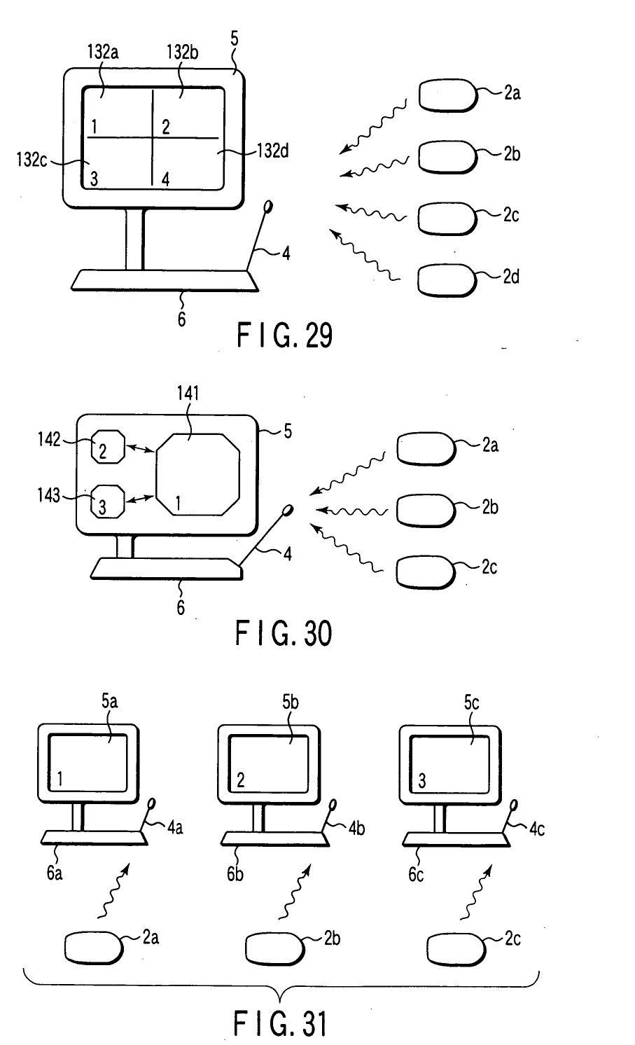

FIG. 29 shows a seventh embodiment of the present invention. The ninth embodiment is different from the first embodiment in terms of the endoscope system 1.

That is, in the seventh embodiment, a plurality of (four in this embodiment) divided segment image screens 132a, 132b, 132c and 132d are provided on one monitor 5 in a way to correspond to four capsule endoscopes 2a, 2b, 2c and 2d and four images of the four capsule endoscopes are displayed on the respective segment image screens at a time. At this time, since signals of different frequencies are sent to the fourth capsule endoscopes 2a, 2b, 2c and 2d, there occurs any cross-talk therebetween.

FIG. 30 shows an eighth embodiment of the present invention. The eighth embodiment is different from the first embodiment (FIGS. 1 to 9) in terms of the endoscope system 1 as will be set out below.

That is, in the eighth embodiment, a main image screen 141 and a plurality of sub-image-screens 142 and 143 are provided on one monitor 5. Here, out of the three capsule endoscopes 2a, 2b and 2c, one capsule endoscope 2a is used to display a corresponding image on the main image screen 141 and the remaining two sub-image-screens 142 and 143 are used to display corresponding images. The display of the main image screen 141 and those of the sub-image-screens 142 and 143 can be arbitrarily switched by an associated switch.

FIG. 31 shows a ninth embodiment of the present invention. The ninth embodiment is different from the first embodiment (see FIGS. 1 to 9) in the endoscope system 1 as will be set out below.

The ninth embodiment includes, for three capsule endoscopes 2a, 2b and 2c, corresponding independent receivers 4a, 4b and 4c, monitors 5a, 5b and 5c connected to the receivers 4a, 4b and 4c and consoles 6a, 6b and 6c, respectively.

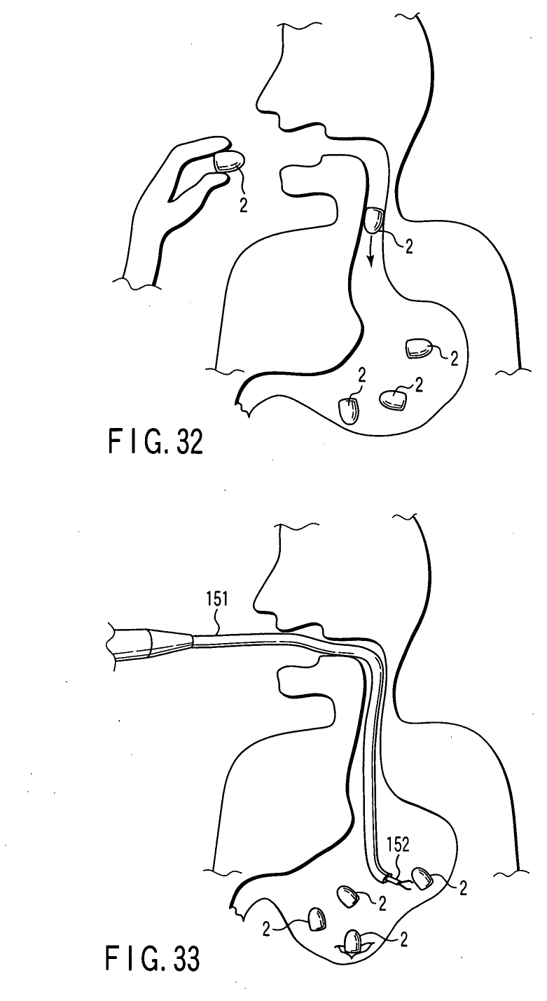

FIGS. 32 and 33 show a tenth embodiment of the present invention. The tenth embodiment is different from the first embodiment in terms of the structure of the endoscope system 1 as will be set out below.

That is, the tenth embodiment constitutes a modified method for inserting a plurality of capsule endoscopes 2 into a body cavity of a patient. Here, the patient swallows a plurality of capsule endoscopes 2 as shown in FIG. 32 into the body cavity.

After this as shown in FIG. 33, an endoscope 151 is inserted into the body cavity of the patient to fix the capsule endoscope 2 to the inner wall of the body cavity by a clip forceps 152.

In this case, it is not necessary to first insert/withdraw the endoscope 151 into/out of the body cavity of the patient. The operation involved can be easily done.

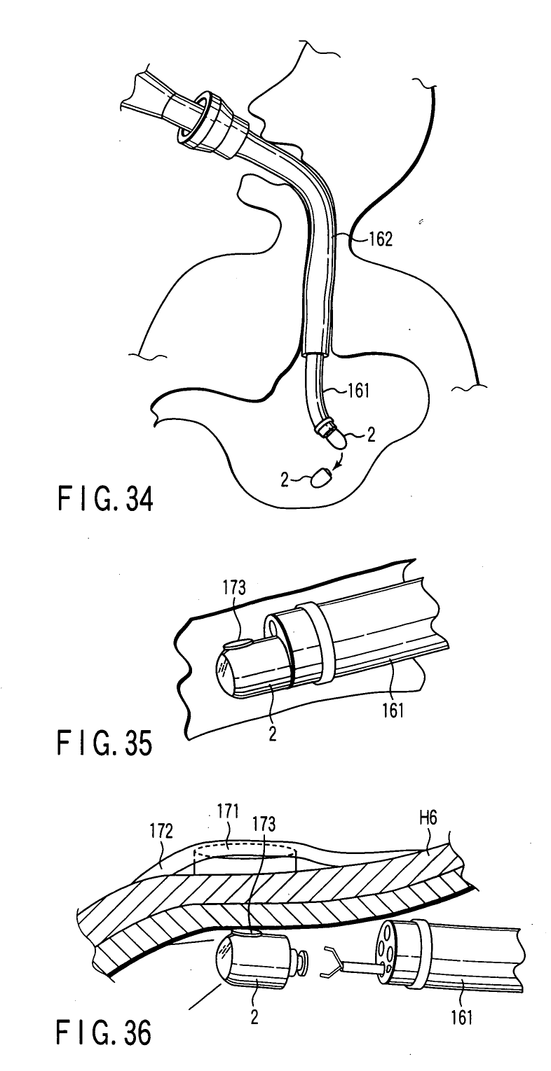

FIG. 34 shows an eleventh embodiment of the present invention. This embodiment is different from the first embodiment of the present invention in terms of the endoscope system 1 as will be set out below.

That is, the eleventh embodiment constitutes a modified method for inserting a capsule endoscope 2 into a body cavity of the patient. Here, the capsule endoscope 2 engaged on the distal end of the endoscope 161 is dropped down onto the inner cavity at the time of inserting the endoscope 161 and the dropped capsule endoscope 2 is detained on the inner wall of the body cavity.

Further, use is made of an overtube 162 and, by repetitively inserting/withdrawing the endoscope 61 beyond the overtube 162 into/out of the body cavity, a plurality of capsule endoscopes 2 are dropped in the body cavity and fixed by the clip fixing device 152 (see FIG. 33) to the inner cavity of the patient.

In this case, it is effective to positively position the capsule endoscopes in the vicinity of a predetermined region of interest.

FIGS. 35 and 36 show a twelfth embodiment of the present invention. This embodiment is different from the first embodiment (see FIGS. 1 to 9) in terms of the endoscope system as will be set out below.

In the twelfth embodiment, as shown in FIG. 36, a magnet 171 is fixed to an outer side of the patient. This magnet 171 is fixed to an abdominal wall H6 of the patient, for example, by an adhesive tape 172.

Further, the capsule endoscope 2 has a fixing section 173 on its outer peripheral surface. The fixing section 173 is made of at least any one of a magnet and a magnetic material, such as a metal, and is fixed in place under the magnetic force of the magnet 171.

The sequence of using the system will be explained.

As FIG. 4 shows, the capsule endoscope 2 is coupled to the distal end of the endoscope 17. Then, the surgeon inserts the insertion section 18 into a body cavity, while observing the interior of the cavity through the capsule endoscope 2. When the capsule endoscope 2 reaches the desired region, it is disconnected from the endoscope 2. Subsequently, the clip fixing device 28 is guided to the capsule endoscope 2 through a channel of the endoscope 17 as shown in FIG. 5. The surgeon manipulates the clip fixing device 28, holding the capsule endoscope 2 and orientating the view field thereof in a desired direction. Thereafter, as shown in FIG. 9, the narrow-diameter scope 47 is guided through one of the lumens of the three-lumen overtube 43. Then, the surgeon inserts the distal end portion of the scope 47 into the body cavity, while observing the interior of the cavity through the scope 47. The overtube 43 is inserted into the body cavity along the insertion section of the endoscope. Next, as FIG. 8 shows, the scope 47 is pulled from the lumen of the overtube 43, leaving the overtube 43 in the body cavity. Treating tools are inserted into the body cavity through the lumens of the overtube 43. The surgeon can move the treating tools to desired regions in the body cavity, while observing these regions through the view field of the capsule endoscope 2.

Not the overtube 43, but a guide wire may be inserted into the body cavity through the lumen made in a treating tool as is illustrated in FIG. 21. In this case, the wire can guides the treating tool into the body cavity. The guide wire may be introduced into the body cavity by a method known in the art. That is, an endoscope having a lumen for guiding the wire is inserted into the body cavity, the guide wire is inserted into the body cavity rough the lumen, and the endoscope is pulled from the body cavity, leaving only the guide wire in the body cavity.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An endoscope system comprising:
a treating device which is configured to perform a treatment in a body cavity and which has an insertion section to be inserted into the body cavity through an opening to the body cavity, said insertion section having a distal end portion and a proximal end portion; and
an observation device which has at least an outer case and an observation optical system and which is used in combination with the treating device.

2. The endoscope system according to claim 1, wherein the treating device has no integrally incorporated observation functions.

3. The endoscope system according to claim 2, wherein a distal end of the insertion section has only an opening of a tube which guides the treating device into the insertion section.

4. The endoscope system according to claim 1, wherein the treating device has a support section which supports the observation device at the distal end of the insertion section, removably from the distal end thereof.

5. The endoscope system according to claim 1, further comprising a support mechanism which supports a device capable of moving the observation device when controlled by a device provided outside the body cavity.

6. The endoscope system according to claim 1, wherein the observation device has a function of switching a close view field to a distant view and vice versa.

7. The endoscope system according to claim 1, wherein the observation device includes a plurality of observation units which have differ in optical performance.

8. The endoscope system according to claim 1, wherein the observation device has a plurality of observation units, and a monitor system is provided, which can display images supplied from the observation units at the same time or at least one selected one of the images.

9. The endoscope system according to claim 1, wherein the outer case of the observation device has a holding section which secures the observation device to a body wall.

10. A method, wherein an observation device and an observation-device retaining device are simultaneously or independently inserted through an opening into a body cavity, the observation device is held at a desired position by using the observation-device retaining device, and a treating device is inserted through the opening into the body cavity to perform treatment.

11. The method according to claim 11, wherein the observation-device retaining device is common to an endoscope or the treating device.

12. The method according to claim 11, wherein the treating device is inserted into the body cavity, by using, as an insertion guide, a guide wire or an overtube inserted into the body cavity into the body cavity through the observation-device retaining device or the endoscope.
